(19) United States
(12) Patent Application Publication
(10) Pub. No.: US 2025/0003007 A1
(43) Pub. Date: Jan. 2, 2025

(54) DNA METHYLATION SIGNATURES FOR PREDICTING RESPONSE TO IMMUNOTHERAPY

(71) Applicant: H. LEE MOFFITT CANCER CENTER AND RESEARCH INSTITUTE INC., Tampa, FL (US)

(72) Inventor: Xuefeng Wang, Tampa, FL (US)

(21) Appl. No.: 18/697,752

(22) PCT Filed: Oct. 4, 2022
(86) PCT No.: PCT/US2022/077520

§ 371 Date: Apr. 2, 2024

Related U.S. Application Data
(60) Provisional application No. 63/251,896, filed on Oct. 4, 2021.

(51) Int. Cl. C12Q 1/6886

(52) U.S. Cl. CPC C12Q 1/6886; C12Q 2600/106; C12Q 2600/154; C12Q 2600/158

(57) ABSTRACT

Introduced herein is the concept of tumor-based expression quantitative trait methylation (cQTM) for prioritizing and systematic mining of predictive biomarkers. With melanoma as a disease model, cQTM CpGs and genes are shown to represent new and efficient candidate targets to be investigated for both prognostic and immune status monitoring purposes.

### CROSS-REFERENCE TO RELATED APPLICATIONS

This application claims benefit of U.S. Provisional Application No. 63/251,896, filed Oct. 4, 2021, which is hereby incorporated herein by reference in its entirety.

### STATEMENT REGARDING FEDERALLY SPONSORED RESEARCH OR DEVELOPMENT

This invention was made with Government Support under Grant Nos. DE030493 and awarded by the National Institutes of Health. The Government has certain rights in the invention.

### BACKGROUND

Detailed characterization of tumor associated immune signatures has become a focus and an integral part of molecular data analysis in cancers that are immunogenic. Immunogenic cancers with high tumor-infiltrating lymphocytes (or immune “hot”) often display distinct mutational profiles, pathogenic mechanisms as well as patient prognosis. DNA methylation signatures in tumors could serve as reliable and archival-tissue-accessible biomarkers for tracking the epigenetic dynamics shaped by both cancer cells and tumor microenvironment. However, it remains challenging to screen across all CpGs sites for identifying the most promising marker panels, given the ultra-high-dimensionality and non-collapsible nature of the data.

### SUMMARY

Introduced herein is the concept of tumor-based expression quantitative trait methylation (eQTM) for prioritizing and systematic mining of predictive biomarkers. With melanoma as a disease model, eQTM CpGs and genes are shown to represent new and efficient candidate targets to be investigated for both prognostic and immune status monitoring purposes. Three cis-eQTM CpGs (cg07786657, cg12446199, and cg00027570) were strongly associated with and can serve as surrogate biomarkers to the tumor immune cytolytic activity score (CYT). In addition, analysis discovered multiple eQTM genes that can be further exploited for predicting immunoregulatory phenotypes. Importantly, through a targeted gene panel analysis, one eQTM in TCF7 (cg25947408) was identified as a candidate biomarker for uncoupling overall T cell differentiation and exhaustion status in a tumor. The prognostic significance of this eQTM, as an independent signature to CYT, was validated by both TCGA and Moffitt melanoma cohort data.

Therefore, disclosed herein is a method for predicting response of a solid tumor in a subject to an immunotherapy agent that involves detecting in a sample from the subject methylation levels of cg07786657, cg12446199, and cg00027570; and calculating a cytolytic activity score (CYT) score from the methylation levels, wherein a high CYT score (CYT^{high}) is an indication that the tumor will respond to an immune checkpoint inhibitor.

In some embodiments, the CYT score is calculated according to the formula:

$$CYT = a - b ( cg 07786657 ) + c ( cg 12446199 ) - d ( cg 00027570 ) ,$$

wherein a is 0 to 10,
wherein b is 2 to 3,
wherein c is 0.5 to 1.5,
wherein d is 1 to 2.

In some embodiments, the CYT score is calculated according to the formula: CYT=6.31-2.11× (cg07786657) +0.96× (cg12446199)-1.28× (cg00027570), Also disclosed is a method for predicting survival of a subject with a tumor, that involves detecting reduced TCF7 levels (TCF7^{low}), LAG3 levels (LAG3^{low}), HAVCR2 levels (HAVCR2^{low}), TIGIT levels (TIGIT^{low}), or a combination thereof, in a sample from the subject compared to a control, or detecting elevated methylation levels of cg25947408 in the sample from the subject indicative of lower TCF7 levels (TCF7^{low}), LAG3 levels (LAG3^{low}), HAVCR2 levels (HAVCR2^{low}), and/or TIGIT levels (TIGIT^{low}) in the tumor, wherein elevated methylation is an indication of lower TCF7 levels (TCF7^{low}), LAG3 levels (LAG3^{low}), HAVCR2 levels (HAVCR2^{low}), and/or TIGIT levels (TIGIT^{low}) in the tumor and a good prognosis.

In some embodiments, this method further involves detecting in the sample methylation levels of cg07786657, cg12446199, and cg00027570; and calculating a cytolytic activity score (CYT) score from the methylation levels, wherein a high CYT score (CYT^{high}) and low TCF7 levels (TCF7^{low}), LAG3 levels (LAG3^{low}), HAVCR2 levels (HAVCR2^{low}), and/or TIGIT levels (TIGIT^{low}) is an indication of a good prognosis.

Also disclosed is a personalized method for treating a tumor a subject that involves detecting in a sample from the subject methylation of cg07786657, cg12446199, and cg00027570; calculating a cytolytic activity score (CYT) from the methylation levels; and administering an immunotherapy agent to the subject.

Also disclosed is a personalized method for treating a tumor a subject that involves detecting reduced TCF7 levels (TCF7^{low}), LAG3 levels (LAG3^{low}), HAVCR2 levels (HAVCR2^{low}), TIGIT levels (TIGIT^{low}), or a combination thereof, in a sample from the subject compared to a control, or detecting elevated methylation levels of cg25947408 in the sample from the subject indicative of lower TCF7 levels (TCF7^{low}), LAG3 levels (LAG3^{low}), HAVCR2 levels (HAVCR2^{low}), and/or TIGIT levels (TIGIT^{low}) in the tumor; and administering an immune checkpoint inhibitor to the subject. In some embodiments, this method further involves detecting in the tumor tissue methylation levels of cg07786657, cg12446199, and cg00027570; and calculating a high cytolytic activity score (CYT^{high}) score from the methylation levels.

In some embodiments, the sample is a tumor tissue sample. In some embodiments, the sample is a plasma sample. In some embodiments where the sample is plasma, a deconvolution method (e.g. EpiSCORE) is used to distill and optimize the methylation signature. As we discussed, it won't change the eQTM biomarkers we have discovered, but it need more advanced analytics to improve the prediction. And the section of C.3.5 is the machine learning approaches my lab has developed to facilitate the biomarker discovery. Those can be added.

In some embodiments, the tumor of the disclosed methods is a melanoma, head and neck cancer, colon cancer, stomach cancer, lung adenocarcinoma, lung squamous cell carcinoma, uterine cancer, glioma, cervical cancer, breast cancer, bladder cancer or colorectal cancer.

In some embodiments, the immunotherapy agent is an immune checkpoint inhibitor, such as an anti-PD-1 antibody, anti-PD-L1 antibody, anti-CTLA-4 antibody, or a combination thereof.

Also disclosed is a kit containing detection probes or primers configured to detect methylation of cg07786657, cg12446199, cg00027570, and cg25947408.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show characterization of 9921 eQTMs (Spearman's ρ<−0.3 or >0.3) in cutaneous melanoma based on the TCGA SKCM cohort. FIG. 1A contains two pie charts showing the distribution of melanoma eQTM-neg (with negative correlation between CpG and corresponding gene, i.e. ρ<−0.3) CpG sites in terms of gene region and CpG island relationship, respectively. FIG. 1B shows top 30 eQTM-neg CpGs ranked by correlation coefficients in melanoma. Among them, 9 eQTM genes also harbor eQTM-pos CpGs. The green bars indicate the magnitude of the negative correlations and the orange bars show the magnitude of the positive correlations. FIG. 1C is a Forest plot showing the hazard ratios of top prognostic eQTM CpG (for predicting patient overall survival) and their corresponding gene information. FIG. 1D is a Reacfoam plot of top prognostic eQTM genes listed shows that the selected genes are highly enriched in immune system-related pathways.

FIGS. 2A to 2C show identification of cis-eQTMs that are predictive of tumor cytolytic activity score. FIG. 2A contains scatterplots and correlations (Pearson) between seven cis-eQTM CpGs and tumor CYT score (*** indicates the p-value for a correlation is <0.001). The seven CpGs were selected from six CYT genes: CD2, CD247, CD3E, GZMA, NKG7, and PRF1. FIG. 2B shows feature importance scores generated from gradient boosting machine (trained based on the seven cis-QTM CpGs) shows that three CpGs (cg07786657, cg12446199 and cg00027570) provides the most informative biomarker panel in terms of predicting CYT. FIG. 2C shows the strong predictive value of the three CpGs can be validated in an independent melanoma dataset (data extracted from GSE144487).

FIGS. 3A to 3E show robust high-dimensional biomarker selection method identifies candidate eQTMs for immunophenotyping of tumors. FIG. 3A is a flowchart showing the resampling based high-dimensional biomarker selection scheme for selecting most reliable eQTM biomarker set in predicting tumor immune phenotypes. FIG. 3B shows the performance of final predictive models shows that eQTMs provides better prediction of IIS and TIS than of APM and ISG.RS scores. All models became nearly saturated after ˜30 biomarkers are included. FIG. 3C contains volcano plots displaying the top predictive eQTM genes (predicting IIS). The x-axis is the average effect size (beta) in the final models (representing predictability) and y-axis indicate how many times that one eQTM was selected after 200 resampling training (representing reliability). FIG. 3D contains volcano plots displaying the top predictive eQTM genes (predicting TIS). The x-axis and y-axis are the same as defined in 3C. FIG. 3E, left panel shows the top eQTM genes and overlapped ones for predicting IIS and TIS. FIG. 3E, right panel shows the top eQTMs in the prediction of APM and ISG.RS scores. The overlapped genes are labeled by red and green colors, respectively. The genes with strong correlations between CpGs and their corresponding gene expression value are highlighted in bold. The positive-correlation eQTMs (or eQTM-pos) are labeled by asterisk.

FIGS. 4A to 4E show eQTM signature in TCF7 as a biomarker for uncoupling overall T cell differentiation. FIG. 4A shows a principal component analysis loading plot of 13 existing biomarkers for T cell exhaustion and naïve T cell state. PCA was performed on gene expression values (TPM) from the TCGA SKCM cohort. The PCA loading plot shows how strongly each gene correlates or influences principal components. The angels between two vectors inform how these genes correlate with each other, with a small angel indicating highly positive correlation and a 90-degree angle indicating independence. For example, GE values of PDCD1 and HAVCR2 are highly correlated in melanoma tumors. As expected, the first principal component (x-axis) shown in the graph is dominated by overall T cell infiltration. The second principal component (y-axis) is a possible indicator for T cell differentiation state, with the first quadrant containing all T cell exhaustion genes and the fourth quadrant containing all naïve T cell related genes. FIG. 4B is a scatter plot of cg259477408 (eQTM in TCF7) methylation value vs gene expression (log2 TPM) value of TCF7 and PDCD1. FIG. 4C shows identification of four patient subgroups for eQTM-based signatures for T cell states: TCF7highCYThigh (Group I), TCF7lowCYThigh (Group II), TCF7lowCYTlow (Group III), and TCF7highCYTlow (Group IV). FIG. 4D shows survival analysis of TCGA SKCM patients stratified according to the CYT-TCF joint eQTM signatures. FIG. 4E shows prognostic significance of TCF7 eQTM (based on methylation value of cg259477408) across 33 TCGA cancer types. The estimated hazard ratios are based on the Cox regression models for each individual cancer type after adjusting for the CYT signature (eQTM).

FIGS. 5A to 5C show validation and application of the TCF7 eQTM signature in Moffitt melanoma cohort. FIG. 5A is a flowchart of Moffitt melanoma sample being selected for the EPIC array methylation analysis. FIG. 5B shows alignment of estimated T cell and B cell infiltration of Moffitt samples with all TCGA SKCM samples. X-axis represents the estimated T/B cell infiltration score based on ssGSEA, and Y-axis represents the eQTM (methylation) based estimation of T/B cell infiltration. The gray dots are TCGA samples and the solid dot represent 30 Moffitt samples. FIG. 5C shows survival analysis of Moffitt melanoma patients stratified according to the CYT-TCF joint eQTM signatures (The group ID and colors are the same as defined in FIGS. 4C & 4D).

FIGS. 6A to 6C show characterization of eQTM-pos in cutaneous melanoma based on the TCGA SKCM cohort. FIGS. 6A and 6B contain two pie charts showing the distribution of the melanoma eQTM-pos (with positive correlation between CpG and corresponding gene, i.e. ρ>0.3) CpG sites, in terms of gene region and CpG island relationship, respectively. FIG. 6C shows top 30 eQTM-pos CpGs ranked by correlation coefficients in melanoma. The orange bars indicate the magnitude of the positive correlations and the green bars show the magnitude of the positive correlations if there is eQTM-neg in the gene region.

FIG. 7 shows fit criteria from best subset regression analysis shows that a 3-CpG panel reaches sufficient precision in predicting the CYT score in melanoma. The model was fit using seven candidate eQTM (CpGs listed in Table 2) in CYT related genes. The plot was generated using the function ols_step_best_subset in R package “olsrr”.

FIGS. 8A and 8B containsvolcano plots displaying the top predictive eQTM genes for APM and ISG.RS signatures. The x-axis is the average effect size (beta) in the final models (representing predictability) and y-axis indicate how many times that one eQTM was selected after 200 resampling training (representing reliability), same as in FIG. 3C and 3D.

FIG. 9 shows the PCA loading plot for principal components 2 (x-axis) and 3 (y-axis) (see FIG. 4A). The PCA loading plot shows how strongly each gene correlates or influences each principal component.

FIG. 10 is a schema of utilizing cfDNA methylation profiles as noninvasive liquid biopsy biomarkers.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “CpG” refers to a dinucleotide sequence, wherein a cytosine nucleotide occurs next to a guanine nucleotide in the linear sequence of bases along its length. In a CpG sequence, the cytosine nucleotide is 5′ to the guanine nucleotide, and the two nucleotides are connected by a phosphate molecule. Cytosines in CpG dinucleotides can be methylated to form 5-methylcytosine. In mammals, methylation of the cytosine within a gene or promoter can affect transcriptional regulation of the gene. Enzymes that add a methyl group are called DNA methyltransferases. The term “CpG” island refers to a genomic region that contains a high frequency of CpG sites.

The term “methylation” refers to the addition of a methyl group to the 5′ carbon of the cytosine base in a deoxyribonucleic acid sequence of CpG within a genome.

The term “methylation status” refers to the presence or absence of a methylated cytosine base at a CpG site.

The term “Methylation assay” refers to any assay for determining the methylation state of one or more CpG dinucleotide sequences within a sequence of DNA.

Disclosed herein are methods of assessing a subject's cancer treatment and prognosis. In some embodiments, the method comprises detecting DNA methylation status at one or more CpG sites disclosed herein in a tumor sample from the subject.

DNA methylation may be detected by any method known in the art, including methylation-specific PCR, whole genome bisulfite sequence, the HELP assay and other methods using methylation-sensitive restriction endonucleases, ChIP-on-chip assays, restriction landmark genomic scanning, COBRA, Ms-SNUPE, methylated DNA immunoprecipitation (MeDip), pyrosequencing of bisulfite treated DNA, molecular break light assay for DNA adenine methyltransferase activity, methyl sensitive Southern blotting, methylCpG binding proteins, mass spectrometry, HPLC, and reduced representation bisulfite sequencing.

In some embodiments methylation is detected at specific sites of DNA methylation using pyrosequencing after bisulfite treatment and optionally after amplification of the methylation sites. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray. Microarrays, and massively parallel sequencing, have enabled the interrogation of cytosine methylation on a genome-wide scale (Zilberman D, Henikoff S. 2007. Genome-wide analysis of DNA methylation patterns. Development 134(22): 3959-3965). Genome wide methods have been described previously (Deng, et al. 2009. Targeted bisulfite sequencing reveals changes in DNA methylation associated with nuclear reprogramming. Nat Biotechnol 27(4): 353-360; Meissner, et al. 2005. Reduced representation bisulfite sequencing for comparative high-resolution DNA methylation analysis. Nucleic Acids Res 33(18): 5868-5877; Down, et al. 2008. A Bayesian deconvolution strategy for immunoprecipitation-based DNA methylome analysis. Nat Biotechnol 26(7): 779-785; Gu et al. 2011. Preparation of reduced representation bisulfite sequencing libraries for genome-scale DNA methylation profiling. Nat Protoc 6(4): 468-481).

The most comprehensive, highest resolution method for detecting DNA methylation is whole genome bisulfite sequencing (WGBS) (Cokus, et al. 2008. Shotgun bisulphite sequencing of the Arabidopsis genome reveals DNA methylation patterning. Nature 452(7184): 215-219; Lister, et al. 2009. Human DNA methylomes at base resolution show widespread epigenomic differences. Nature 462(7271): 315-322; Harris, et al. 2010. Comparison of sequencing-based methods to profile DNA methylation and identification of monoallelic epigenetic modifications. Nat Biotechnol 28(10): 1097-1105).

To detect DNA methylation, a preferred embodiment provides for first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. Preferably, isolated DNA is treated with sodium bisulfite (NaHSO3) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore after PCR or sequencing, the resultant product contains cytosine only at the position where 5-methylcytosine occurs in the starting template DNA. This makes the discrimination between unmethylated and methylated cytosine possible.

The tumor sample may be a solid tumor, such as carcinomas, sarcomas and lymphomas. In some embodiments, the solid tumor is selected from adrenocortical carcinoma, bone tumors, brain cancer, breast cancer, cervical cancer, colorectal carcinoma, desmoid tumors, desmoplastic small round cell tumors, endocrine tumors, esophageal cancer, Ewing sarcoma family tumors, gastric cancer, germ cell tumors, head or neck cancer, hepatoblastoma, hepatocellular carcinoma, lung cancer, melanoma, mesothelioma, nasopharyngeal carcinoma, neuroblastoma, non-rhabdomyosarcoma soft tissue sarcoma, osteosarcoma, ovarian cancer, pancreatic cancer, prostate cancer, retinoblastoma, rhabdomyosarcoma, skin carcinoma, testicular cancer, thyroid carcinoma, uterine cancer and Wilms tumors. The tumor sample may be a hematological cancer, such as leukemia.

In some embodiments, the method further involves treating the subject with an anti-tumor agent. The antitumor agent is selected from an angiogenesis inhibitor, such as angiostatin K1-3, DL-α-Difluoromethyl-ornithine, endostatin, fumagillin, genistein, minocycline, staurosporine, and (+)-thalidomide; a DNA intercaltor/cross-linker, such as Bleomycin, Carboplatin, Carmustine, Chlorambucil, Cyclophosphamide, cis-Diammineplatinum (II) dichloride (Cisplatin), Melphalan, Mitoxantrone, and Oxaliplatin; a DNA synthesis inhibitor, such as (t)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriccin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, Dolastatin 15, Nocodazole, Paclitaxel, Podophyllotoxin, Rhizoxin, Vinblastine, Vincristine, Vindesine, and Vinorelbine (Navelbine); and an unclassified antitumor agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-a, Rapamycin, Sex hormone-binding globulin, Thapsigargin, and Urinary trypsin inhibitor fragment (Bikunin). The antitumor agent may be a monoclonal antibody such as rituximab (Rituxan®), alemtuzumab (Campath®), Ipilimumab (Yervoy®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 131l-tositumomab, ado-trastuzumab emtansine, lapatinib (Tykerb®), pertuzumab (Perjeta™), ado-trastuzumab emtansine (Kadcyla™), regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatinib dimaleate (Gilotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), pembrolizumab (Keytruda®),), carfilzomib (Kyprolis®), Ramucirumab (Cyramza™), Cabozantinib (Cometriq™), vandetanib (Caprelsa®), Optionally, the antitumor agent is a neoantigen. The antitumor agent may be a cytokine such as interferons (INFs), interleukins (ILs), or hematopoietic growth factors. The antitumor agent may be INF-α, IL-2, Aldesleukin, IL-2, Erythropoietin, Granulocyte-macrophage colony-stimulating factor (GM-CSF) or granulocyte colony-stimulating factor. The antitumor agent may be a targeted therapy such as toremifene (Farcston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). The antitumor agent may be a checkpoint inhibitor such as an inhibitor of the programmed death-1 (PD-1) pathway, for example an anti-PD1 antibody (Nivolumab). The inhibitor may be an anti-cytotoxic T-lymphocyte-associated antigen (CTLA-4) antibody. The inhibitor may target another member of the CD28 CTLA4 Ig superfamily such as BTLA, LAG3, ICOS, PDL1 or KIR. A checkpoint inhibitor may target a member of the TNFR superfamily such as CD40, OX40, CD137, GITR, CD27 or TIM-3. Additionally, the antitumor agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zolinza), Romidepsin (Istodax), or Ruxolitinib (Jakafi).

To explicitly address the heterogeneity issue of DNAme in bulk tissues, deconvolution (e.g. EpiSCORE algorithm) can be used to enable the in silico decomposition of methylomes. The algorithm first uses existing single-cell RNA-sequencing datasets and the anticorrelation relationship between DNAme and gene expression to construct a reference DNAme-atlas. The cell-type-specific methylation signals can then be estimated based on the weighted robust linear multivariate model. The current EpiSCORE implementation provides estimated relative abundance of 40 cell types (defined based on 13 tissue types). Immune scores, such as T cell infiltration score (TIS), overall immune infiltration score (IIS), can be calculated based on these cell-type-specific estimates. The deconvoluted score of other immune hallmarks and immune-related functional scores can also be calcualted, including T cell exhaustion (TCE) score, interferon stimulated genes resistance signature (ISG.RS) score, and tertiary lymphoid structure (TLS) score. All the calculated scores can be tested for their association with survival outcomes and various clinical variables such as smoking, HPV status, and molecular subtypes. As a validation step, the DNAme-derived immune scores can be compared with the scores calculated from the matched transcriptome data based on deconvolution tools including but not limited to CIBERSORTx, Ecotyper, and xCell.

A series of statistical and machine learning methods can be be applied to further prioritize DNAme biomarkers (at both CpG level and region level) and composite final predictive models. These method include (1) resampling elastic net (re-EN): The re-EN is an extension to the random lasso method, developed for high-dimensional feature selection. Each time re-EN randomly selects a subset of variables and a subset of all samples for training with the elastic net method, and then repeats this process for 200 times. In the second step, the most frequently selected biomarkers can be considered for post-selection linear regression (OLS) modeling. The same procedure can be applied to both binary and survival outcomes. (2) Multiple kernel learning (MKL): MKL methods can be applied to evaluate the prediction performance based on all screened DNAme biomarkers. MKL is a promising multi-view learning technique where heterogeneous input data/views are integrated into a single model and each view is represented by a kernel. Our group has implemented the algorithm in C++ and built into an R package “RMKL” (available on CRAN) and have recently extended it to model survival outcomes. (3) Extreme Gradient Boosting (XGBoost): Based on the method XGBoost, the prediction performance of prioritized DNAme markers can be built and assessed in terms of predicting tumor immunity outcomes and patient survival outcomes.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Tumor eQTM screening reveals distinct CpG panels for deconvolving cancer immune signatures

### Material and Methods

A total of 471 TCGA skin cutaneous melanoma tumor samples with matched methylation and gene expression profiles were downloaded from the Broad's GDAC Firehose portal. For methylation profiles, we used the level 3 CpG site (normalized) methylation from the Illumina Infinium HumanMethylation450 platform (450K), which contains 485,577 probes. CpG sites that were missing for more than 5% of samples were excluded. The rest missing CpGs were imputed based on the K Nearest Neighbor method via the impute. knn function from the R package ‘impute’. To normalize the distribution of methylation data and to stabilize the variance, we consider two commonly used transformation methods for beta values (1) M-value transformation: M-value=log2(Beta/(1-Beta)) and (2) arcsine-based transformation: arcsin(2*Beta-1). We used arcsine-based transformation in all high-dimensional regression models performed in this study because it achieves better variance stabilization (Park, Y. and Wu, H. Bioinformatics, 2016 32:1446-1453). For gene expression profiles, the Level 3normalized RSEM values were multiplied by 10⁶ to obtain transcripts per million (TPM), followed by log2(x+1) transformation. Normalized DNA methylation (beta) values, identified via EPIC array, of 196 samples in Lund melanoma cohort were downloaded from GEO with the accession number GSE144487. RNAseq-based gene expression profiles, generated from overlapped fresh frozen melanoma tumors in the same cohort, were downloaded from GEO using the accession number GSE65904.

In this study, we focus on local or cis-eQTMs. In this study, we define cis-eQTMs as those CpG sites located within the 1000 bp of a targeted gene region and they are strongly correlated with the gene expression value. Specifically, for each gene, we calculate the Spearman correlation coefficient between the gene expression (GE) value and each CpG (M value) within the gene region, spanning from 1000 bp upstream and 1000 bp downstream of the gene region (based on gene start and end positions defined in the hg19 build). In each gene region, only CpG sites with the highest negative and positive CpG-GE correlation coefficients were retained. We only used CpGs with a moderate to strong paired correlation (Spearman's ρ<−0.3 or >0.3), resulting in 9921 eQTMs in 8619 genes in the TCGA SKCM dataset.

This study employed a series of well-established gene expression signatures to calculate immune phenotypes in melanoma samples. Cytolytic activity (CYT), a robust measurement of tumor immune competence mediated by cytotoxic T cells (Rooney, M.S., et al. Cell, 2015 160:48-61), was calculated as the geometric mean of two genes GZMA and PRF1. Then, we calculated the overall immune infiltration score (IIS), the T cell infiltration score (TIS) and antigen presenting machinery score (APM) of all tumor samples based on the single sample gene set enrichment analysis (ssGSEA), following the procedure described previously (Şenbabaoğlu, Y., et al. Genome biology, 2016 17:1-25). Finally, we also applied ssGSEA to calculate the interferon-stimulated gene signature ISG.RS, which was found in associated with resistance to immune checkpoint blockade (Benci, J. L., et al. Cell, 2019 178:933-948. e914). ssGSEA signature scores were estimated using the gsva function implemented in the R package GSVA (Hänzelmann, S., et al. BMC bioinformatics, 2013 14:1-15).

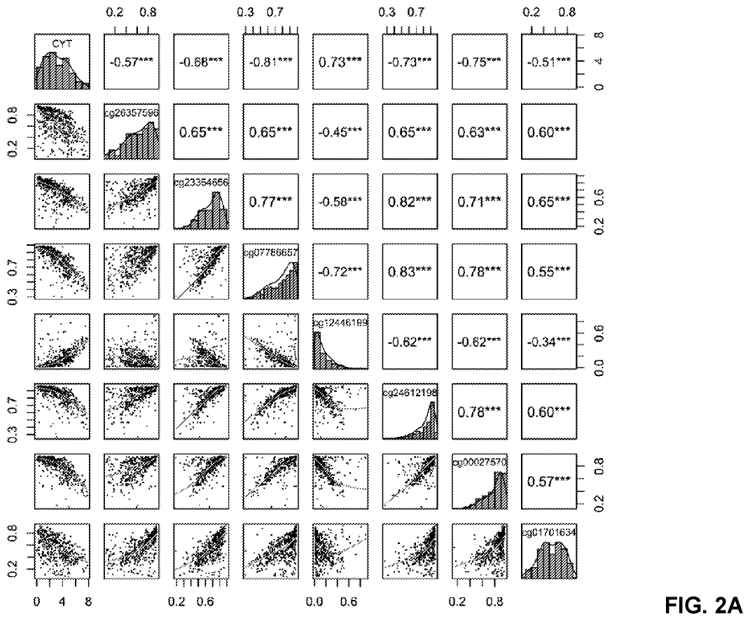

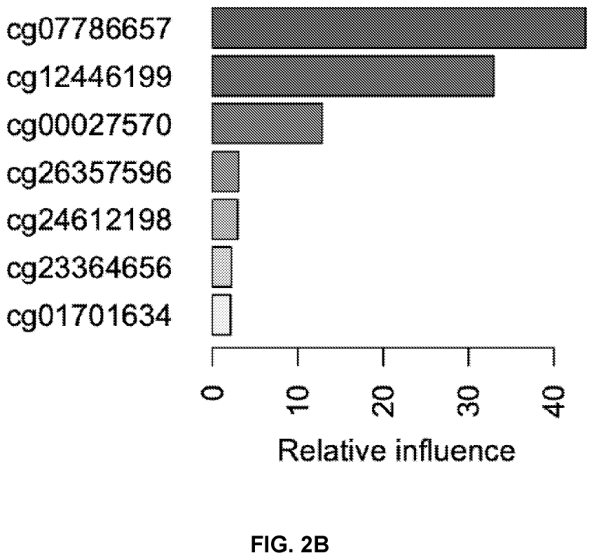

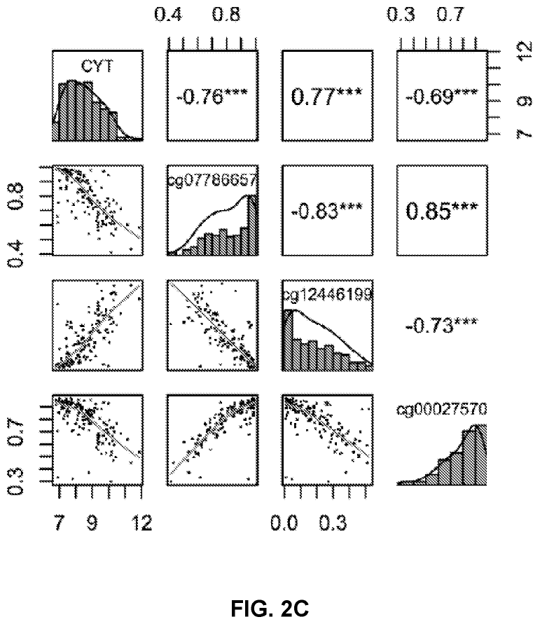

In order to obtain the most robust eQTM panels underlying the above-described immune signatures, a two-step multi-marker building strategy is proposed as depicted in FIG. 2A. In the first step, the random or resampling elastic net method for selecting most predictive eQTMs is applied. The resampling elastic net is an extension to the random lasso method (Wang, S., et al. The annals of applied statistics, 2011 5:468), proposed for high-dimensional feature selection. Similar to random lasso, our algorithm randomly selects a subset (80%) of variables and randomly selects a subset (80%) of all samples for efficient training with the elastic net method, and then repeats this process for B=200 times. In the second step, the most frequently selected eQTM biomarkers are then considered for post-elastic-net linear regression (OLS) modelling and gene-based analysis. The predictive performance of the OLS models is evaluated based on top 10, 20 and 30 eQTMs, respectively. This reasoning is based on an interesting observation in similar projects with TCGA data analysis, where most molecular-marker-based predictive models for predicting immune and survival outcomes become saturated (based on testing errors in cross-validation) when approximately 20 markers are included, regardless of sample sizes. Assuming each predictor requires 10 degrees of freedom, a multivariable regression model can be reliably fitted on the effective sample size is more than 200 subjects. For an external validation dataset, focus is needed on well-performed predictive models with medium to large effect sizes. With a significance level (alpha) of 0.05 and 20 predictors, a sample size of 62 achieves 80% power to detect an effect size (f²) of 0.49 (or R²=0.33) using an F-test, and around 90% power to detect an effect size of 0.59 (or R²=0.37). A systematic search of the existing data (until Jul. 1 2021) in the GEO database showed that the matched samples in GSE144487 and GSE65904 are the only publicly available cohort that is potentially powered for the biomarker validation in melanoma.

### Results

eQTM Landscape in Melanoma

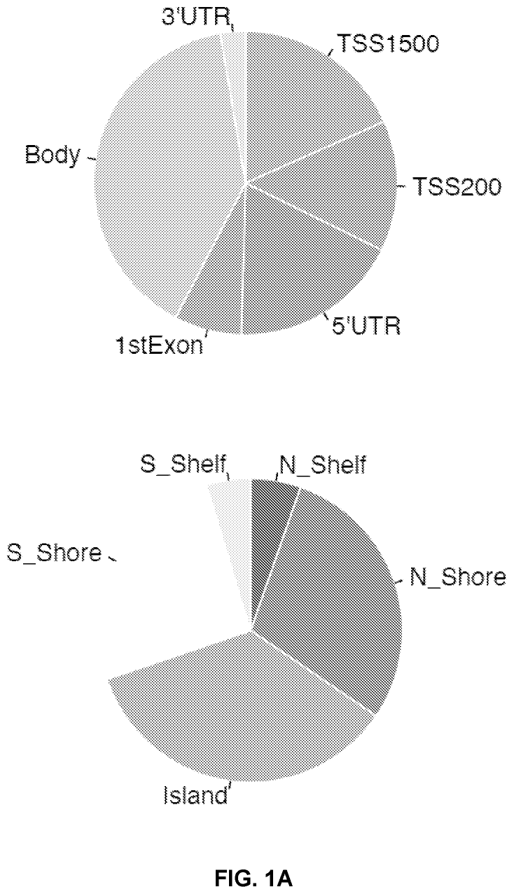

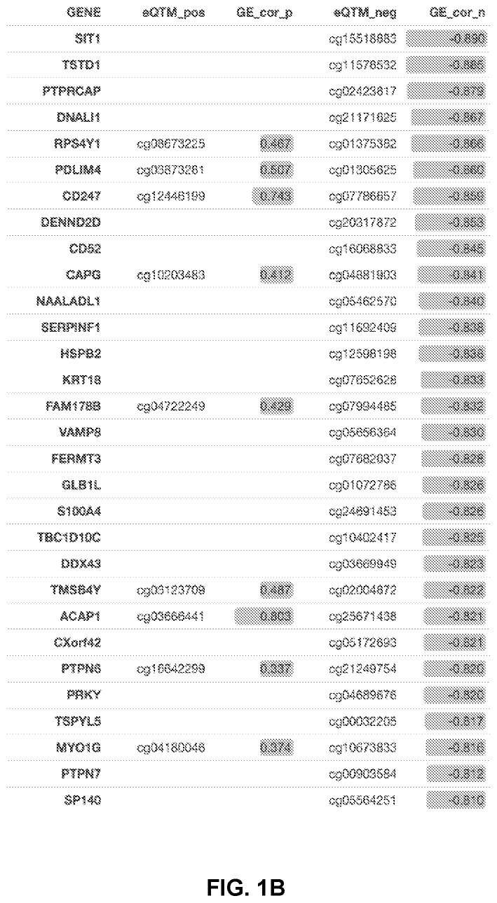

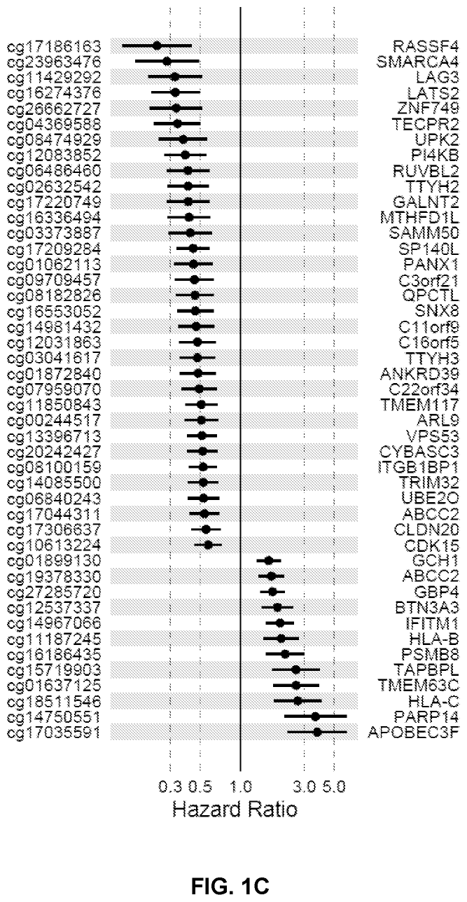

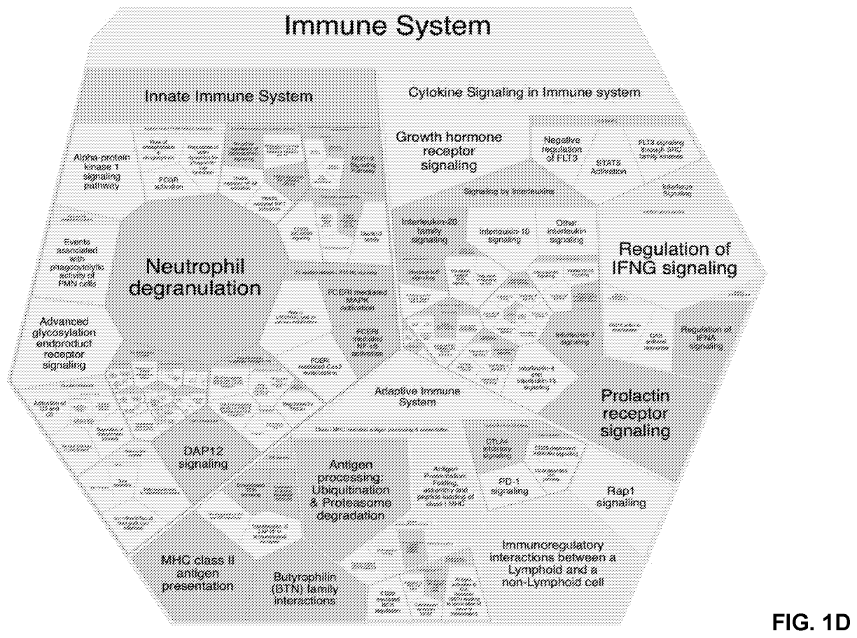

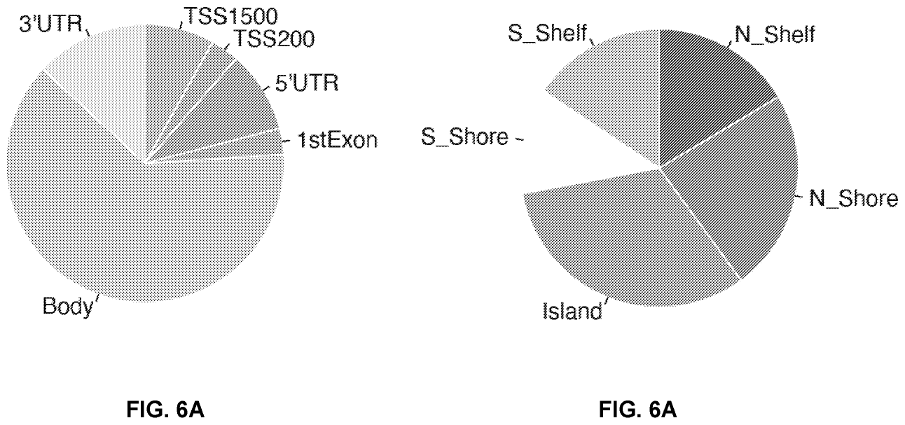

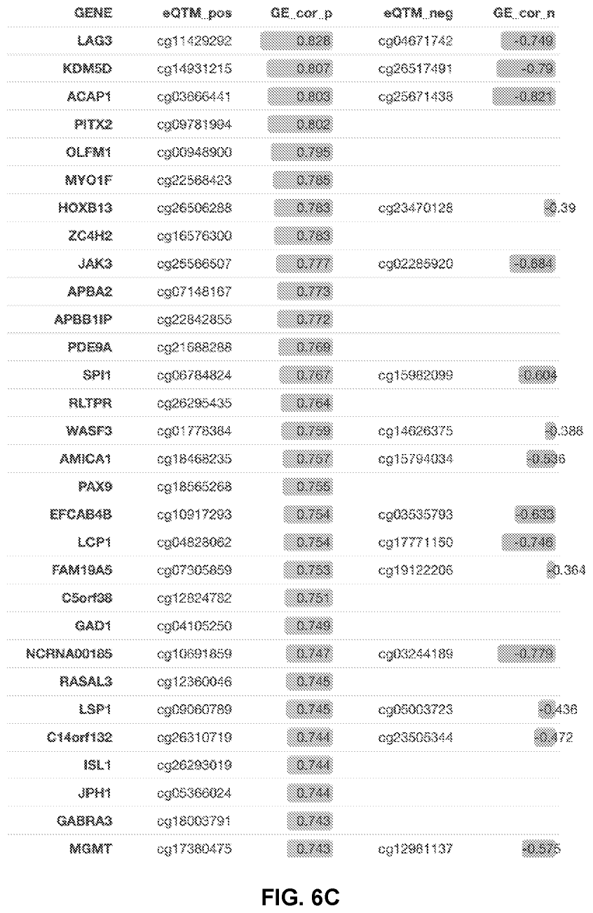

The identified eQTMs were first summarize based on gene-level locations, together with the CpG island annotation. As expected, more eQTMs (6479 out of 9921) showed negative correlations between CpG methylation and gene expression values (CpG-GE) than positive correlations. For simplicity, these are referred to herein as eQTM-neg (and similarly eQTM-pos for positive correlation). The gene location distributions of these eQTM-neg CpGs are represented by the pie charts in FIG. 1A. Remarkably, only about half of the CpGs in eQTM-neg are located in the traditionally-studied promoter regions including TSS1500, TSS200, and 5′UTR. Not surprisingly, a significantly higher proportion of CpGs in eQTM-pos (FIG. 6A) are located in the gene body region. Among all eQTM-neg sites, 737 of them showed strong to intermediate CpG-GE correlations (Spearman's p less than or equal to-0.6), representing the most promising biomarker candidates. The top 30 eQTM-neg CpGs and genes are shown in FIG. 1B (top 30 eQTM-pos in FIG. 6B). CpGs in SIT1 (cg15518883) and LAG3 (cg11429292) are ranked as the top one eQTM-neg and eQTM-pos, respectively. Two TME-related genes, CD247 and ACAP1, harbored both strong eQTM-neg and eQTM-pos, highlighting their potential as sensitive biomarkers for immune profiling in bulk tumors. By recognizing that the high correlations in Y-chromosome genes (such as RPS4Y1 and KDM5D) can be driven by the sex-specific expression, the CpG-GE association was further examined by adjusting for sex. Next, the overall prognostic significance of eQTMs was examined by testing their association with patient overall survival. In univariate Cox's regression, a total of 45 eQTMs showed a P-value surpassing strict Bonferroni correction (˜5×10⁻⁶). The corresponding hazard ratios (HRs) of these top prognostic CpGs and associated genes are shown in FIG. 1C. The

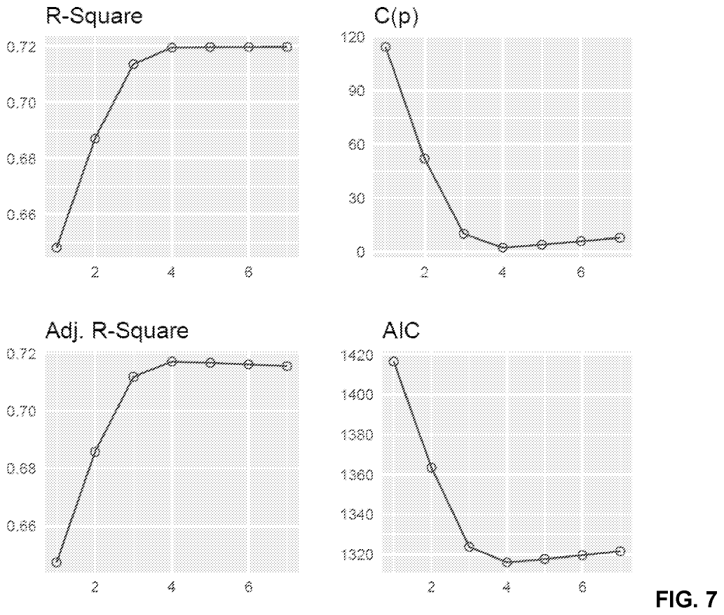

Reactome pathway analysis on these genes indicated that these genes were predominantly enriched in immune system processes, including Adaptive Immune System, Antigen Presentation, Immunoregulatory interactions between Lymphoid and non-Lymphoid cells, and Cytokine Signaling in Immune System (FIGS. 1D and 7). Together, these results suggest that eQTMs in melanoma can encapsulate various type of immunogenic signatures and may provide complementary sources of prognostic markers to assist in clinical management.

cis-eQTM Panel for Predicting Cytolytic Activity

Because cytolytic activity score is the simplest yet robust transcriptome-based immune signature across multiple cancer types (Kalbasi, A. and Ribas, A. Nature Reviews Immunology, 2020 20:25-39), whether the eQTMs within the CYT-related gene regions (cis-eQTM) can be used a methylation surrogate to predict pre-existing anti-tumor immune response was investigated. In the eight CYT genes (including CD2, CD247, CD3E, GZMA, GZMK, GZMH, NKG7, and PRF1), we observed that six genes containing at least one eQTM (Table 1).

TABLE 1

| Gene | eQTM(−) | eQTM(+) |
|---|---|---|
| CD2 | cg00027570 | |
| CD247 | cg07786657 | cg12446199 |
| CD3E | cg24612198 | |
| GZMA | cg26357596 | |
| GZMK | — | |
| GZMH | — | |
| NKG7 | cg01701634 | |
| PRF1 | cg23364656 | |

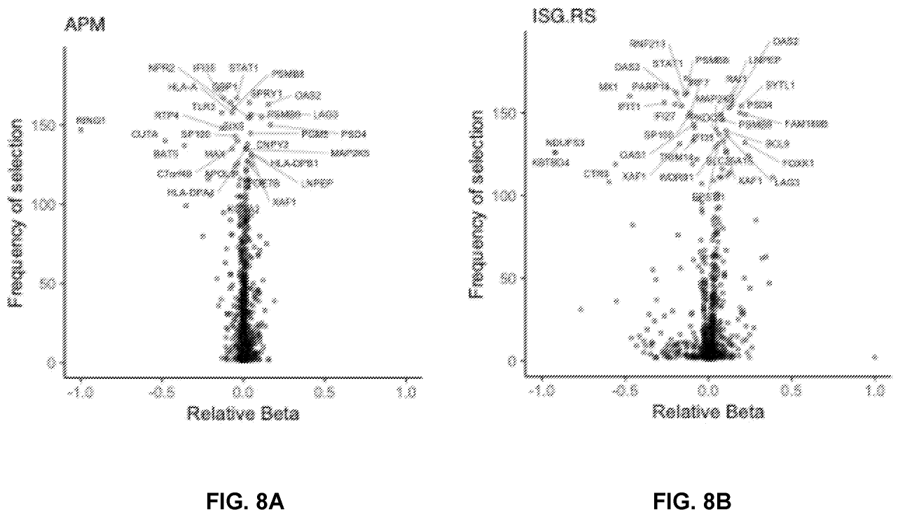

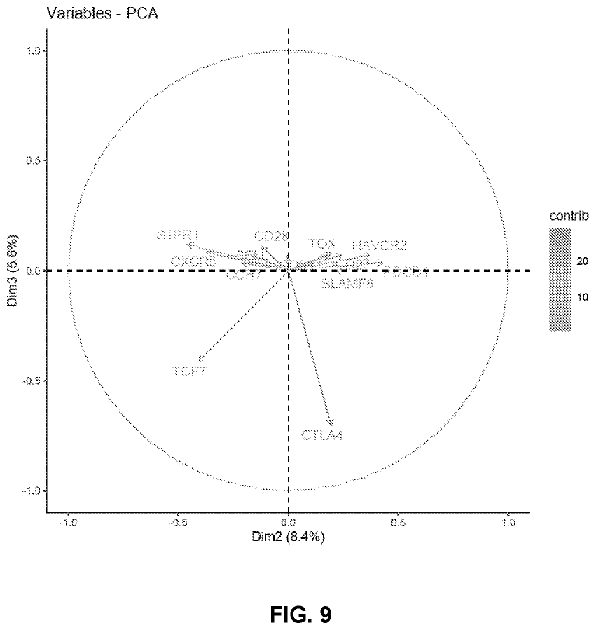

As shown in the pairwise correlation plot (FIG. 2A), all seven eQTMs have a significant negative and positive correlation with CYT score (geometric mean of GZMA and PRF1 GE values). However, compared to other eQTMs in this shortlist, the eQTM in GZMA (cg26357596) and PRF1 (cg233646556) showed a relatively weaker correlation with their corresponding GE values. Both the feature importance score from the gradient boosting (FIG. 2B) and the best subset regression analysis (FIG. 8) indicate that three CpGs (cg07786675 and cg12446199 in CD247, and cg00027570 in CD2) can be used to form a minimal eQTM-based predictive panel. Importantly, the strong correlation between CYT and these eQTMs can be validated in an independent dataset (FIG. 2C) from matched samples in GSE144487 and GSE65904 (the Lund cohort). It is interesting to note that cg07786675 in CD247 is also one of eQTMs possessing the highest correlations (Spearman's p at-0.86) with the corresponding GE value, highlighting its particular relevance in cutaneous melanoma. Using the TCGA SKCM cohort as training dataset, we propose the predictive model of DNAme-based CYT predictive model as follows:

$$CYT = 6.31 - 2.11 \times ( cg 07786657 ) + 0.96 \times ( cg 12446199 ) - 1.28 \times ( cg 00027570 )$$

Here, all CpG values are arcsine transformed beta values. In summary, above targeted cis-eQTM analyses suggest that eQTMs in CD247 and CD2 are more suitable (than GZMA and PRF1) to be used in the clinical setting-as a simplest DNA-based biomarker panel to assess the intrinsic immunological status of tumors, or as the proxy signature to predict response to immune checkpoint blockade (Van Allen, E.M., et al. Science, 2015 350:207-211).

Identifying eQTMs for Predicting Four Immune Signatures

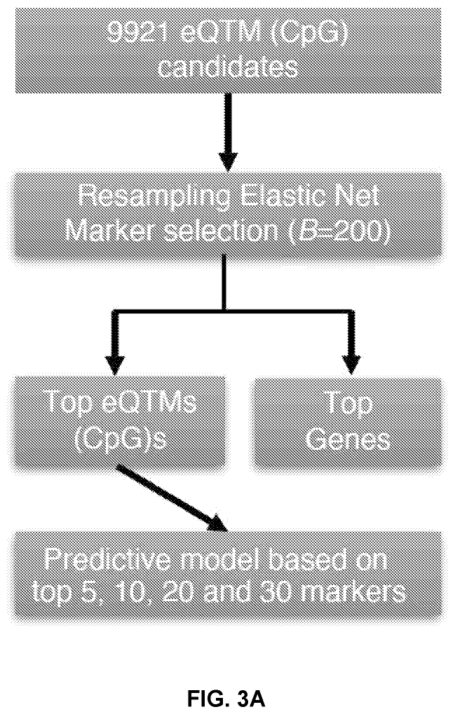

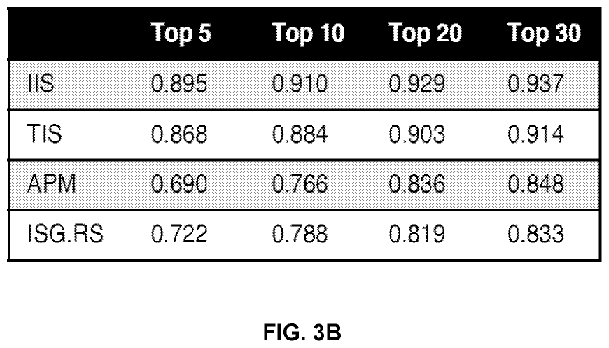

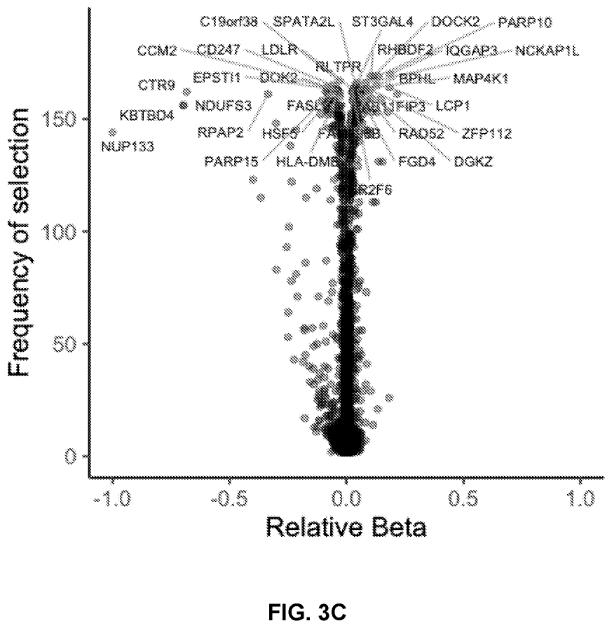

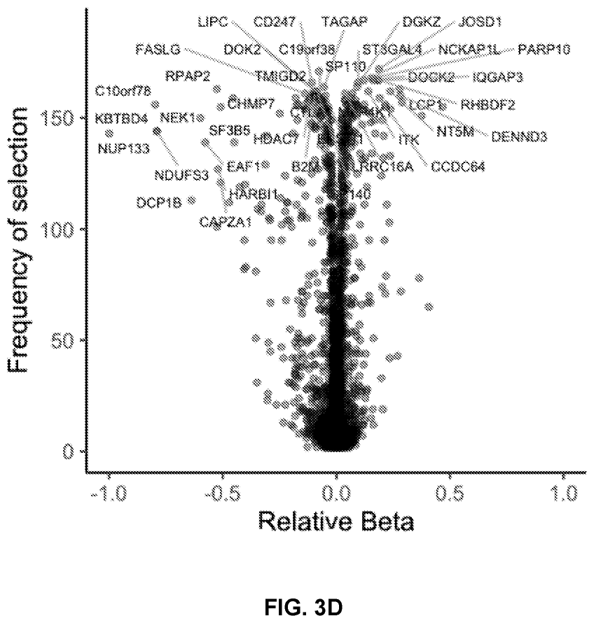

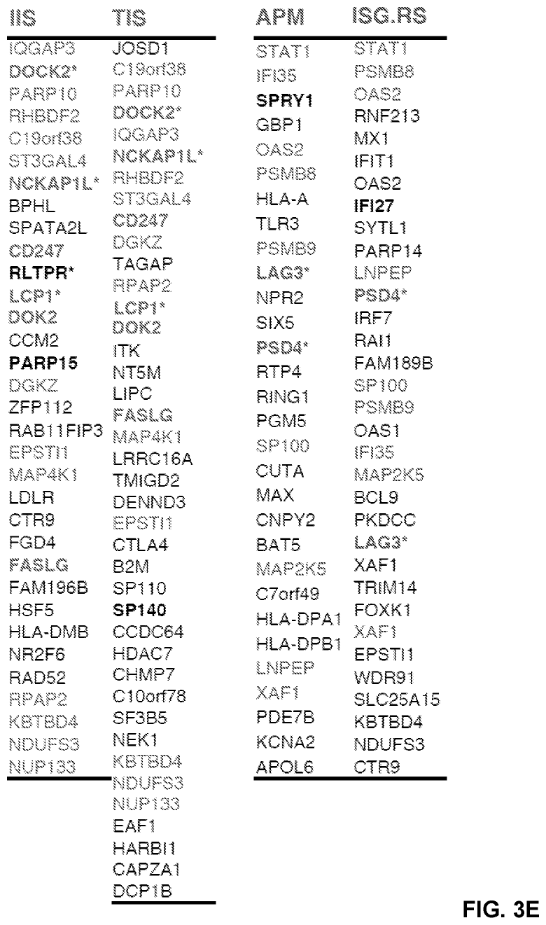

In this section, the predictive potential of eQTMs for systematic immune profiling of tumors was explored. four established immune infiltration and signature scores calculated based on transcriptome, i.e., TIS, IIS, APM and ISG.RS, were investigated. To generate a robust panel of eQTMs, a resampling based high-dimensional biomarker selection pipeline was applied as described in Methods (illustrated in FIG. 3A). Instead of only relying on the eQTMs in signature genes included in calculating these immune scores, the aim is to uncover all predictive biomarkers from the 9921 eQTMs identified in the previous step. Results from evaluating the performance of final predictive models showed that (FIG. 3B) all models achieved near optimal accuracy when top 20 eQTMs were included. Overall, the prediction accuracy on APM and ISG.RS (R>0.8) are relatively less accurate than the prediction on IIS and TIS scores (R>0.9). This is expected because mechanistic signatures are generally more difficult to estimate than cell composition score due to less-differentiated biomarker panels. In addition to finding surrogate methylation sites, eQTM based biomarker screening has important implications for identifying potential functional roles of the underlying genes that have been underappreciated. By labelling the eQTMs with gene annotations in the biomarker selection volcano plots (importance score versus effect size), the most influential genes that are involved in modulating the studied immune signatures can be identified. For example, the volcano plots from the biomarkers for predicting IIS and TIS (FIG. 3C. and FIG. l D) revealed not only multiple immune genes that have been well studied, but also genes with their functions remaining largely elusive in the field of melanoma. the top biomarkers and the overlapped gene panels are highlighted in FIG. 3E. These lists further emphasize the vital role of methylation status in genes such as DOCK2, NCKAP1L, CD247, LCP1 and FALSG in determining the T cell and immune cell infiltrations, as well as genes such as STAT1, LAG3, PSD4, SPRY1 and IFI27 in terms of their pronounced roles in regulating antigen presenting and interferon gamma signaling. In summary, this analysis demonstrates that the genome-wide eQTM screening is a feasible, reliable and efficient approach for building predictive models for various tumor-intrinsic immune signatures, which is particularly useful when RNAseq experiment is more challenging (e.g. in examining archival tumor samples). The top eQTMs, with their close links to the underlying genes, also provides novel and alternative insights into potentially new mechanistic or therapeutic targets for future interventions.

eQTMs to Uncouple T Cell State from Cytolytic Score

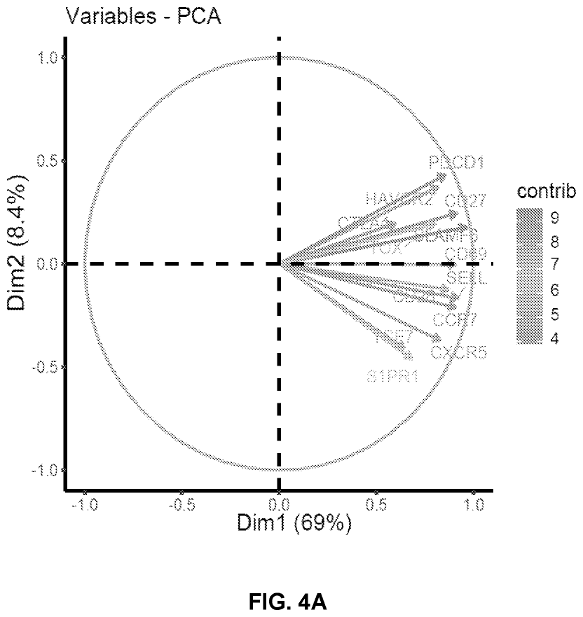

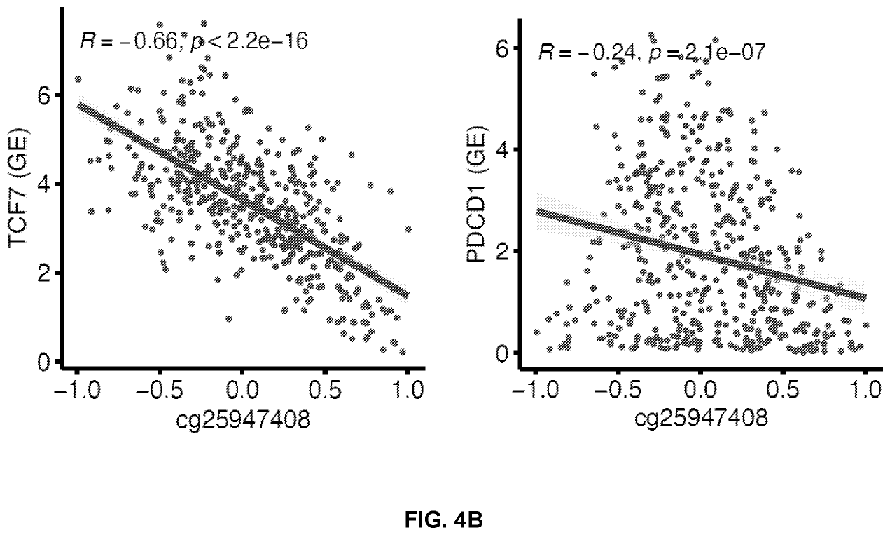

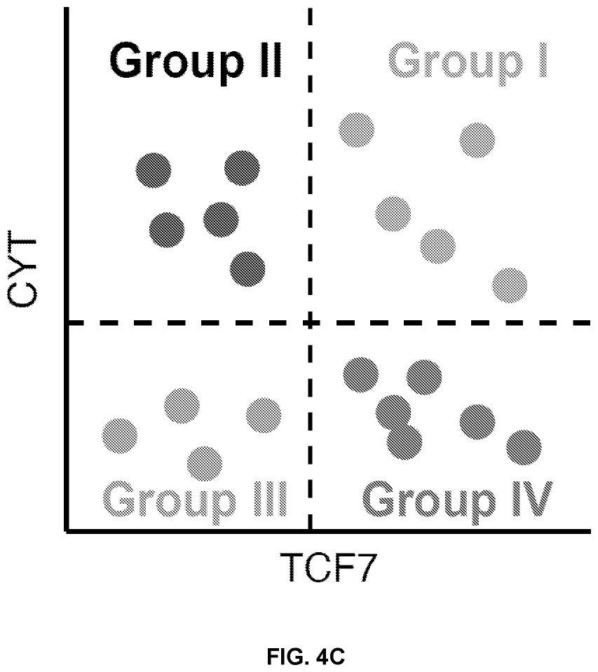

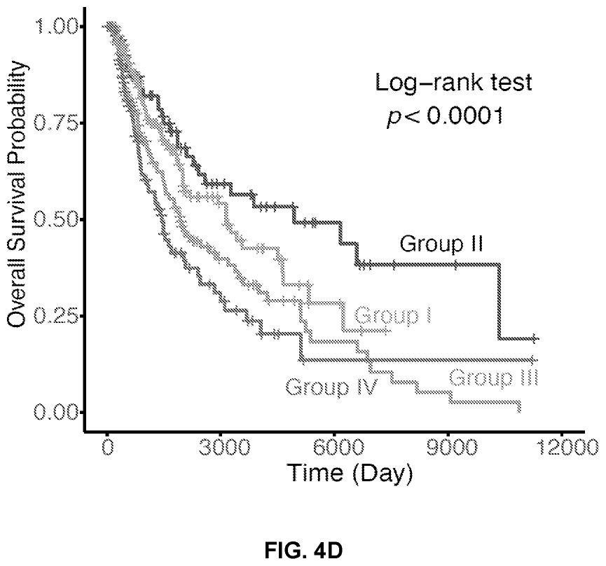

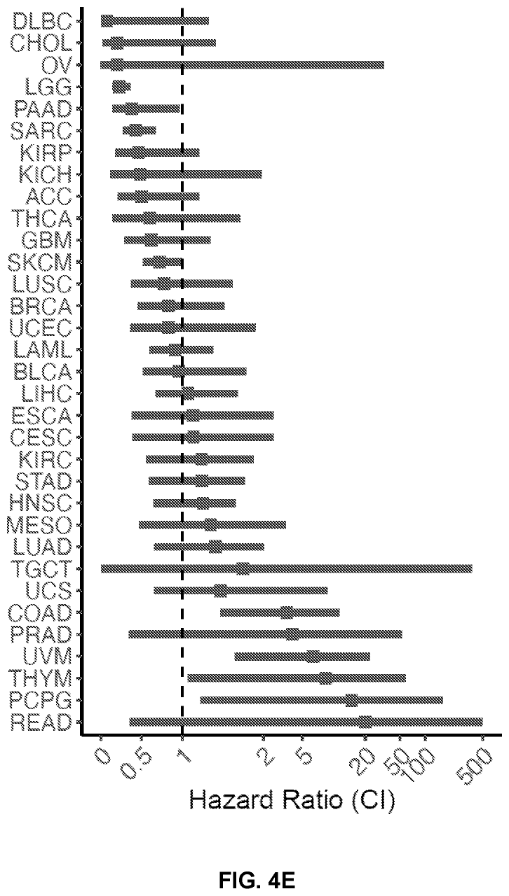

Given the promising results above, next examined was whether eQTMs can be leveraged to infer the T cell differentiation state in TME. Despite the fact that multiple biomarkers have been identified to predict T cell exhaustion and other cell state-related phenotypes, it remains a major challenge to disentangle the significant confounding effects from cellular composition—if only gene expression data from the bulk tumors is used. It is almost an unavoidable dilemma that gene expression of many T cell exhaustion biomarkers such as PDCD1 and HAVCR2 (TIM3) are often highly correlated, and confounded, with generic T cell lineage genes such as CD8A in tumors. To better dissect and understand such correlation structure, Principal Component Analysis (PCA) analysis was performed using gene expression data on 13 most established T cell 2:1001-1012), including PDCD1, CTLA4, HAVCR2, CXCR5, TOX, SLAMF6, CD69, TCF7, CCR7, SELL, S1PR1, CD27, and CD28. The PCA loading plot (FIG. 4A) shows the correlation strength of each gene with the first two principal components (PCs). The plot also illustrates the relationship between any two genes, where a smaller angle between the loading vectors indicates more positively correlated. Hence, PC1 (explains 69% of the total variance) can be safely interpreted as representing an overall immune cellular score, which is the main shared correlation component among all immune marker genes. There was an interesting phenomenon in the direction of the PC2 (y-axis): all six genes in the first quadrant of the PCA loading plot are known biomarkers for

T cell exhaustion progression, while the genes in the fourth quadrant in the loading plot are known marker genes reflecting naïve T cell state. This finding suggests that PC2 may represent an overall T cell precursor state, determined by relative abnundances between native and activated T cells in different stages. Based on the PCA loading plot, we can infer that CXCR5, TCF7 and S1PR1 are genes most orthogonal with PDCD1 and HAVCR2, and therefore may provide more specific signatures on predicting T cell states. In these three genes, only TCF7 includes an eQTM (cg25947408) with relatively strong CpG-GE correlation. As expected, cg25947408 showed much higher correlation with TCF7 gene expression than PDCD1 (FIG. 4B). Based on above results, it was hypothesized that characterizing tumor samples by incorporating T cell state signatures might allow us to refine the immune response profiling. Specifically, patients can be stratified into four groups: TCF7^{high}CYT^{high} (Group I), TCF7^{low}CYT^{high} (Group II), TCF7^{low}CYT^{low} (Group III), and TCF7^{high}CYT^{low} (Group IV), based on the four quadrants approach as depicted in FIG. 4C. Using the single-eQTM (cg25947408) as a surrogate for the TCF7 signature together with the DNAme-based CYT score as describe previously, we divided the TCGA SKCM patients into four patient groups. To prevent overfitting, median values of CYT and TCF signature score were used as the cutoffs to divide patients. Each patient group thus had similar sample sizes. The Kaplan-Meier plot (FIG. 4D) showed that patients in Group Il had the best overall survival, followed by Group I, Group III, and Group IV. The observed pattern in this plot indicates that the eQTM in TCF7 provides an independent immune prognostic signature and overall higher cg25947408 methylation value (or lower TCF7 signature) is associated with better patient prognosis. Lower TCF7 levels reflects a lower proportion of tumor-reactive T cell vs bystander T cells. As to be discussed in the next section, this trend was also successfully replicated in our own cohort even with very limited sample size. To explore the potential of the TCF7 eQTM as a pan-cancer prognostic biomarker, the Cox regression model was performed with both cg25947408 and eQTM-based CYT score as covariates in 33 TCGA cancer types in which DNA methylation data of the targeted CpGs were available. The cg25947408 methylation was significantly or marginally significantly associated with patient overall survival in multiple other cancer types, most notably in LGG, SARC, COAD and UVM (FIG. 4E). However, higher cg25947408methylation values were found associated with worse outcome in COAD and UVM, indicating the existence of context-dependent role of the overall T cell state in affecting patient prognosis. In summary, the eQTM in TCF7 (specifically, cg25947408) is a potentially independent, robust and specific signature biomarker that is predictive of overall T cell differentiation or exhaustion state in bulk tumor and patient outcomes.

### Illustration of eQTM Signatures using Moffitt Melanoma Cohort

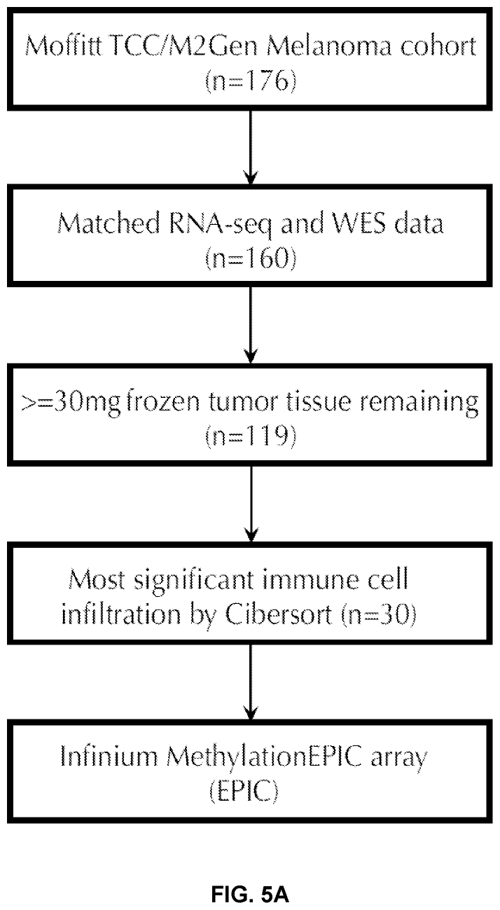

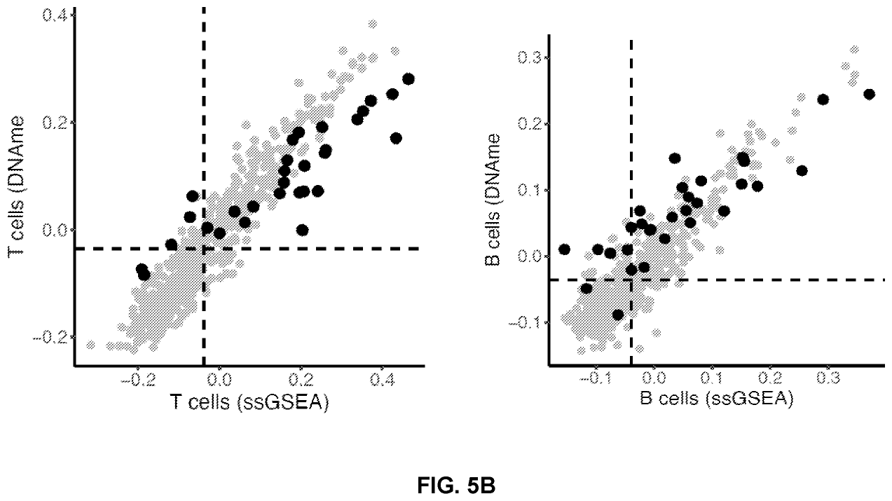

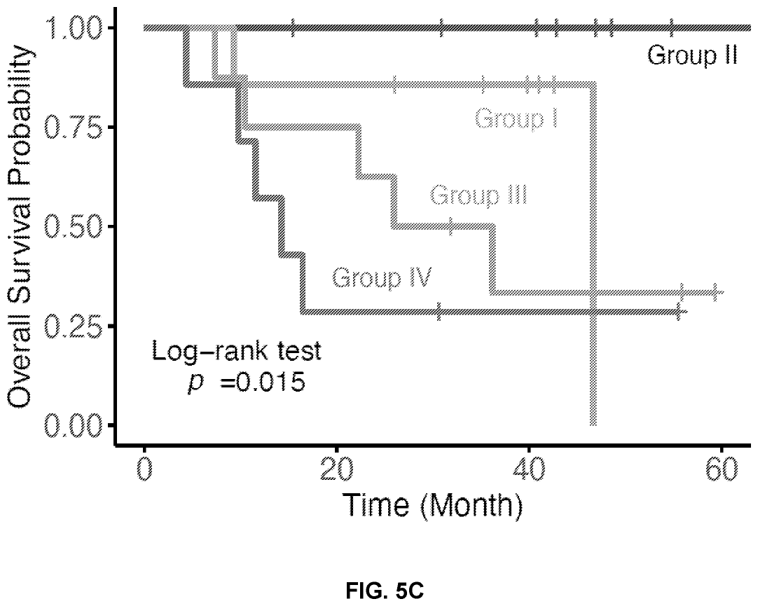

The utility of the above described eQTM signatures was also illustrated and validated based on a high-quality data acquired from a Moffitt melanoma cohort. As depicted in FIG. 5A, a total of 160 patients in this cohort (out of 170) had matched RNA-seq and WES, and 119 patients had >30 mg remaining tumor tissue available for DNA extraction. It was hypothesized that the eQTM signatures will provide additional prognostic information even in the patient subgroup with immune-hot tumors. Therefore, the aim was to prioritize the samples for methylation profiles based on gene-expression deconvolution results, calculated by the CIBERSORT software. We performed EPIC array DNA methylation analysis on top 30 samples with the most significant p-values generated from the CIBERSORT analysis (Newman, A.M., et al. Nature methods, 2015 12:453-457). To minimize the intertumoral heterogeneity, it was ensured that methylation assay and RNAseq were based on DNA and RNA extracted from the same tumor within a patient. The high immune infiltration of these samples can be corroborated by aligning the GE- and DNAme-based infiltrated T cell and B cell measures with the TCGA SKCM samples in the sample plot (FIG. 5B), where most of the Moffitt samples lied in the first quadrant (i.e. high DNAme-and GE-estimated T/B cell scores). Similar to the patient stratification (TCF and CYT) described in the previous section, we separated all patients into four groups and compared their survival outcomes (FIG. 5C). Surprisingly, the previously observed pattern can be replicated even with very limited samples, where the TCF7^{low}CYT^{high} patient subgroup and TCF7^{high}CYT^{low} subgroup exhibited the most and least favorable outcomes, respectively. This case study highlighted the translational potential of the TCF7-CYT joint eQTM signature as a reliable clinical and translation biomarker.

### Discussion

Overall, the tumor DNA methylation landscape provide highly informative, sensitive, and stable epigenetic markers for cancer medicine. Despite that large amount of DNAme data have been generated from bulk tumor tissues in many large-scale cancer datasets, they represent a highly underutilized resources in translational oncology research. One main challenge is that the local levels of DNA methylation are subject to substantial confounding from complicated cellular composition and tumor heterogeneity in bulk tissues. Furthermore, standard biomarker screening methods may yield less reproducible results due to the ultra-high-dimensionality and non-collapsible nature of the measured CpG features. The common practice of filtering CpGs in the non-promoter regions will likely miss many important sites that are more informative for tumor profiling. In this study, the aim was to extend the concept of eQTM to tumor samples and examined their potentials as prioritized epigenetic biomarkers, with particular attention to their ability in elucidating the tumor-immune ecosystem dynamics. To this end, the ability of eQTMs in predicting patient prognosis and a variety of established immune phenotypes was comprehensively studied, with cutaneous melanoma as an immunogenic disease model.

While the concept of eQTM is broadly defined as any CpGs having moderate to strong correlation with any gene expression values, the focus of the study was on the analysis of cis-eQTM (i.e. CpGs located in a targeted gene region). Through genome-wide analysis of cis-eQTM detection, it was shown that top eQTMs (i.e. with highest correlation between gene expression and CpG methylation) were enriched in gene regions associated with immune infiltration and response. It is noteworthy that several genes such as CD247 and ACAP1 harbor CpGs exhibited strong negative and positive correlation with the corresponding gene expression values. On the other hand, the top prognostic eQTMs were highly enriched in genes of immunoregulatory functions as demonstrated by the Reactome pathway analysis. As demonstrated in the prognostic biomarker and the subsequent immune biomarker screening, one key advantage of cis-eQTM is that each CpG is uniquely connected to the corresponding gene expression level with the highest relevance in terms of gene function. In this setting, the identified prognostic CpG, e.g., cg12537337, is not merely a numeric surrogate but also more likely a functional proxy for a gene (BTN3A3) with regard to its predictive value for patient survival. Therefore, although eQTM is essentially a prioritized CpG site, it encapsulates both DNA methylation and the underlying gene expression information.

This analysis revealed that the cytolytic activity score, a well-established tumor immune score, can be reliably predicted by three cis-eQTM methylation sites (cg07786657, cg12446199 and cg00027570) from CYT-related gene regions. Six out of eight CYT genes contain at least one eQTM site, further demonstrating the essential role of DNA methylation in shaping tumor immunity. Two eQTM sites, cg23364656 and cg26357596 were found within genes PRF1 and GZMA, which are the original gene set proposed to quantify the CYT score. Based on a robust resampling-based biomarker selection strategy, this analysis further identified top predictive eQTM biomarker panels for estimating four other immune scores. Most models will get saturated in terms of prediction performance gain when the number of biomarkers included are more than 30, which aligned with our previous experience with methylation biomarkers. In predicting IIS and TIS, we observed that many top eQTMs that can be replicated via resampling-based analysis reside in well-known immunoregulatory genes such as DOCK2, NCKAP1L, CD247, LCP1 and FASLG. Although the prediction accuracy on APM and ISG.RS were less striking compared to T cell deconvolution, our analysis detected several overlapped eQTMs (such as those in PSD4, LAG3, and STAT1) in the top list that warrant further evaluation and functional validation.

Despite single-cell RNAseq being a mainstay of T cell subtype identification in recent cancer research, single-cell sequencing is challenging for translational studies or for archival tumor samples because it is still expensive, low throughput in terms of patient sample size, and requires freshly prepared tissues. Encouraged by the positive results in estimating the above-mentioned immune phenotypes, this study also explored the ability of eQTM as candidate biomarkers for the overall T cell differentiation status in bulk tumors. With the intention to generalize our findings to other cancer types, we focused on searching eQTMs in 13 well-established marker genes for T cell differentiation. As expected, the PCA loading plot demonstrated that all these genes exhibited moderate to strong correlation with the first principal component which represents the overall immune infiltration. The PCA loading plot and subsequent analyses revealed that the eQTM (cg25947408) in TCF7 is the most promising biomarker candidate in melanoma. TCF7 encodes the TCF1 protein, which acts as a transcription factor and histone deacetylase that plays critical role in shaping innate and adaptive immunity (Raghu, D., et al. Trends in immunology, 2019 40:1149-1162). More recent studies have suggested that this gene is a specific and stable biomarker for delineating naive-like and exhausted T cell subtypes (Zhang, J., et al. The FASEB Journal, 2021 35: e21549; Andreatta, M., et al. Nature Communications, 2021 12:1-19; Chen, G. M., et al. Cancer Discovery, 2021). Although TCF7 is expressed in many types of T cells and natural killer cells, it is often expressed at significantly higher level in naïve T cells. In general, a well-accepted hypothesis is that TCF7⁺ tumor-reactive T cells are reservoir that needed to generate effector T cell. Likewise, if using cg25947408 as a DNAme proxy, higher methylation level would be observe at this site in tumors with higher abundance of non-naïve-like T cell types, e.g. exhausted, terminal-effector like, or dysfunctional T cells. In a similar setting, the well-known T cell exhaustion genes such as PDCD1 (which encodes PD-1) are less suitable for bulk tumor deconvolution in melanoma because they are highly confounded by the overall T cell infiltration in the tumor, as indicated by the high correlation between PDCD1 expression and other generic T cell lineage genes. Together, it was reason that the eQTM in TCF7 provides a plausible tool to uncouple the naïve-like and exhaustion-like T cell phenotype in the challenging bulk tumor setting. This possibility was supported by analysis comparing melanoma patient subgroups obtained from four TCF7-CYT quadrants. The TCF7-eQTM signature provides additional prognostic information in both CYT-high and immune-cold CYT-low patient subgroups. Interestingly, higher TCF7 signature (or lower cg25947408 methylation) appeared to be associated with favorable and unfavorable survival in the CYT-high and CYT-low groups, respectively. This result indicates that the relative abundance of naïve-like T cells plays a different prognostic role in patients having immune-hot and immune-cold tumors. Results from the analysis across all major cancer types in TCGA showed that the TCF7-eQTM signature holds the potential to serve as a pan-cancer biomarker. Remarkably enough, we were able to replicate the finding in survival group stratification in a local immune-hot-enriched melanoma dataset even with a limited sample size. Taken together, all these results suggested that the potential of cis-eQTMs (e.g. TCF7) as a novel biomarker for uncoupling T cell state phenotypes and as robust biomarker candidates in the translational and clinical setting.

### Example 2. Liquid Biopsy Biomarkers

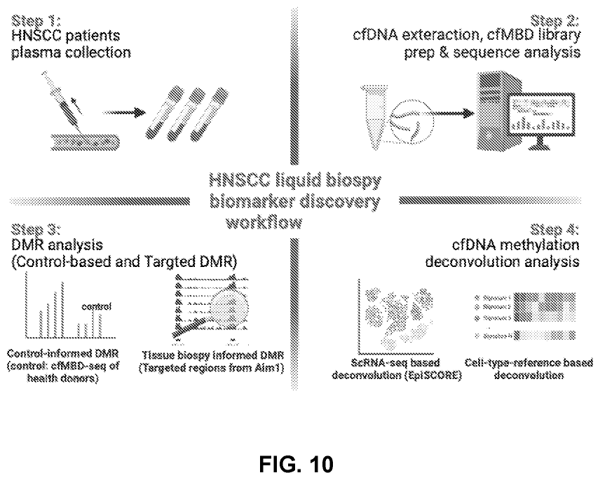

Circulating DNAme has proven to be one of the most promising methods for establishing noninvasive biomarkers, whereby more efficient early-detection, prognostic prediction and serial monitoring of cancer can be implemented. In this Example, plasma cell-free DNA is collected and examined from (n=200) HNSCC patients and systematic bioinformatics analyses performed to discover diagnostic and prognostic biomarkers in HNSCC. In order to effectively use multi-modality information, banked plasma samples are used and plasma samples collected from those patients with (or will have) matched tumor tissue biopsy molecular data. cfMBD-seq, a highly reliable and sensitive DNAme profiling method, is used to uncover cfDNA methylation characteristics. As illustrated in FIG. 10, integrative approaches are used to maximize the power in the biomarker detection by combining data from external sources, including (1) established cfMBD-seq from health donors; (2) identified DMR regions from tissue biopsy for a targeted study; (3) HNSCC scRNA-seq data for a finer deconvolution of cfDNA methylation profiles.

Blood samples (10 cc) are collected in anti-coagulant tubes (EDTA). Within 2 hours after blood draw, whole blood is centrifuged (2000 rpm) at 4° C. for 20 minutes. The supernatant plasma is centrifuged again in the same conditions to collect platelet-poor plasma, then stored immediately at −80° C. until use. cfDNA is extracted using QIAamp Circulating Nucleic Acid Kit (Qiagen; Hilden, Germany) following the manufacturer's protocol, except for the addition of carrier RNA in Buffer AVE. All cfDNA eluates will be quantified by Qubit Fluorometer.

10ng cfDNA is used for end repair/A-tailing and adapter ligation KAPA Hyper Prep Kit (Kapa Biosystems; Wilmington, MA, USA) is used. Adapter ligated DNA is first combined with methylated filler DNA to ensure that the total amount of input for methylation enrichment is 100 ng, which is further mixed with 0.2 ng of methylated and 0.2 ng of unmethylated spike-in A. thaliana DNA from DNA Methylation control package (Diagenode). To capture methylated cfDNA, the adaptor-ligated cfDNA is subjected to methylation enrichment using MethylCap Kit (Diagenode). MethylCap protein will be 10-fold diluted to 0.2 μg/μl using Buffer B. The eluted fraction will be purified by DNA Clean & Concentrator-5 Kit. The purified DNA will be divided into two parts, one for qPCR (PowerUp™ SYBR™ Green Master Mix, Thermo Fisher) amplification of spiked-in DNA for methylation enrichment quality control, another for library amplification. Recovery of the spiked-in methylated and unmethylated controls can be calculated based on cycle threshold (Ct) value of the enriched and unenriched samples. Specificity of the capture reaction can be calculated by (1−[recovery of unmethylated control DNA over recovery of methylated control DNA])×100). The specificity of the reaction should be ≥99% before proceeding to the next step.

Methylation-enriched DNA libraries is amplified for 12 cycles and then pooled for high-output 75 bp single-end read. The sequence reads are then aligned to the human genome (hg38) using Bowtie-2 (Version 2.4.2) [59] with default settings. After the alignment, the generated sam files are converted into bam files, followed by sorting, indexing, removal of duplicate reads, and extraction of read count on chr1-chr22 using SAMtools (Version 1.11) [60] ‘view’, ‘sort’, ‘index’, and ‘markdup’ command lines. R (Version 4.0.3 or greater) package RaMWAS (Version 1.12.0) [61] with default parameters are used for quality control of overall mapping quality and calculation of non-CpG reads percentage, average non-CpG/CpG coverage (noise), and CpG density at peak. CpG annotation reference is obtained from R package annotatr (Version 1.16.0): annots='hg38_cpgs'. BEDtools (Version 2.28.0) [62] ‘coverage’ command line will be used to call the number of sequenced reads on each CpG feature. CpG feature coverage of each sample is combined as a count matrix. Transcripts per kilobase million (TPM) normalization is performed before comparing the percentage of CpG feature coverage between different groups.

Rows with inter CpG regions and 0 read count among all samples are filtered out from CpG feature raw count matrix. Filtered matrix is further subset for single cancer type and non-cancer control and fit a negative binomial model to call DMRs at BH-FDR <0.1 (Wald test) using R package DESeq2 (Version 1.32.0) [63]. R package EnhancedVolcano (Version 1.10.0) [64] is used for visualization of fold change and BH-FDR (q value) for all CpG islands and extended CpG islands. Unsupervised hierarchical clustering is performed on Partek genomics suite (Version 7.0) for visualization of DMRs, using log transformed DESeq2 normalized values, z scores, Euclidean distance, and

Ward Clustering. R package pcaExplorer (Version 2.18.0) [65] is used for principal component analysis of DESeq2 normalized values of top 1,000 DMCGIs selected by highest row variance. The 95% confidence ellipses for the case and control were displayed. DMRs with fold change >2 will be used for intersection with tissue derived DMCs.

Based on the cfDNA methylation profiles, HNSCC-optimized computational approach is applied to facilitate the in silico deconvolution of (1) cfDNA cellular makeup and (2) cfDNA-based immune signatures. Similar to the cellular deconvolution with transcriptome data, methylome-based deconvolution has also emerged as a powerful and reliable technique for characterizing tumoral and cellular heterogeneity. However, existing methods for DNAme deconvolution are mainly focusing on bulk tumor tissues or whole blood sample. In this Example, in silico deconvolution approach is benchmarked and optimized for enrichment-based methylation profiles obtained from plasma cfDNA. Moreover, a more HNSCC-optimized reference is built based on head and neck cancer single-cell RNAseq datasets.

The basic idea of reference-based deconvolution algorithm is to assume that the measured DNAme profile (M) from the heterogeneous tissue or blood samples is generated as the weighted sums of cell-type-specific reference matrix (C), and solving for these weights (P) thus reveals relative proprotions for each cell-type group included

in the reference panels [66]; thus often solved by optimzing min∥C·P−M∥². The traditional method for generating C, based on the orthogonal gene signatures learned from sorted cells or samples with known mixtures, is suboptimal for plasma or tissue samples because their cellular and tissue composition is much more complicated and varied than whole blood samples. A recently developed algorithm called EpiSCORE is used to leverage the single-cell RNAseq datasets to “impute” the DNAme reference matrix C. Briefly, the single-cell expression reference (scGEP) is first generated based on the annotated cell types from three large-scale datasets in HNSCC. The methylation reference matrix is then imputed based on those marker genes in which promoter DNAme and GE level is anticorrelated. The markers in the methylation reference is futher filtered by focusing on regions that can be reliably detected by cfMBD-seq. Finally, P is solved using the Huber's robust M-estimator in the weighted robust partial correlation (wPRC) framework. As a comparison, the deconvolution is also performed based on the reference matrix derived from the purified cell types. The ability of the calculated signature in distinguishing the plasma samples from HNSCC patients and health donors is systematically tested. The prognostic value of these signatures is also tested by correlating them with patient survival groups.

### Example 3. Predictive Value of Selected Cancer-Specific and Immune-Specific DNAme as Potential Prognostic Biomarkers for Immunotherapy Outcomes in HNSCC Patients

In this Example, the predictive value of selected cancer-specific and immune-specific DNAme are evaluated as potential prognostic biomarkers for immunotherapy outcomes in HNSCC patients. It is hypothesize that addition of a targeted panel of immunoregulatory cfDNAme biomarkers to existing liquid biopsy biomarkers can improve the patient stratification strategies toward personalized immune checkpoint Inhibitors treatments. To accomplish this goal, circulating cfDNA (plasma samples) we are collected and examined from a total of 60 HNSCC patients before and after receiving immunotherapy (pembrolizumab/nivolumab). In order to create an effective balanced design (the anti-PD-1/PD-L1 objective response rate of HNSCC is lower than 20%), approximately 30 patients are selected from the responder (complete response and partial response) group and 30 patients from the non-responder group (progressive disease and stable disease). the ultra-sensitive cfMBD-seq as described in Example 2 is used for the methylation profiling of plasma samples. The tumor biopsy collected before treatment are also profiled with methylome (based on RRBS) and transcriptome (based on RNAseq). The primary analysis goal is to assess the accuracy gain of predicting immunotherapy outcome by adding the discovered cfDNA methylation biomarkers from the pre-treatment plasma samples only, which allows translational application of our finding to clinics. The secondary goals are to compare the difference of cfDNA methylation profiles before and after treatment and to correlate the DNAme signatures obtained from the tumor and liquid biopsies.

The plasma collection, ctDNA extraction and and cfMBD-seq library preparation steps are similar to the steps described in Example 2. Very briefly, cfDNA is first isolated from 2 ml plasma samples using QIAamp Circulating Nucleic Acid Kit (Qiagen). Library construction is performed using KAPA Hyper Prep Kit to add sequencing adaptors. Lambda DNA is used as a filler to add up to 100 ng for the methylation enrichment. After the methylation enrichment, the enriched DNA fragments are amplified with 9˜12 PCR cycles to add unique index to each sample. All the final libraries are sequenced on the NextSeq 550 platform (Illumina). The Bioinformatics analyses on the sequence data will be conducted similar to those steps described above.

To facilitate a multi-modality comparison, tumor tissue biopsies collected before treatment from the immunotherapy cohort are also interrogated using RNAseq and RRBS. The sequencing and bioinformatics steps are similar to the steps described above. One special limitation is that most tumor biopsies collected from the immunotherapy cohort arenot available as fresh frozen samples but are instead achieved as formalin-fixed paraffin-embedded (FFPE) blocks. The reason is that most HNSCC patients treated by immunotherapy are diagnosed at advanced or metastatic stage; subsequently, it is difficult to retrieve the pre-treatment fresh frozen tissues. One would thus expect some samples yielding poor data quality in RNAseq, but not RRBS data. As explained previously, one important advantage of DNAme profile is that it can be reliably assayed with archival materials.

As a baseline model, the predictive values of existing liquid biomarker panel is first tested (e.g, RASSF1A, SHOX2, SETP9, CDKN2A and MGMT). These biomarkers are selected based on previous publications suggesting their potential association with diagnosis/prognosis in head and neck cancer. the predictive performance of adding the selected and validated DMR signatures from Example 2 are then evaluated and compared. The performance of the optimized and baseline model in terms of predicting immunotherapy response is evaluated using multiple measures including accuracy, sensitivity, specificity and area under the receiver operating characteristics curve (AUC). Special cfDNAme panels: the predictive performance of two special sub-panels is evaluated: (1) panels based on hypermethylation-only biomarkers to test the hypothesis that hypermethylated site can be detected with better sensitivity in plasma; and (2) panels constructed from biomarkers in prioritized functional regions (i.e. immune synapse, SE, and miRNAs) to test the hypothesis that focusing on epigenetically regulated functional regions is also a viable strategy for discovering robust cfDNAme biomarkers.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A personalized method for treating a tumor a subject comprising:
(a) detecting in a sample from the subject methylation of cg07786657, cg12446199, and cg00027570;
(b) calculating a cytolytic activity score (CYT) from the methylation levels wherein a high CYT score (CYT^{high}) is an indication that the tumor will respond to an immunotherapy agent; and
(c) administering an immunotherapy agent to the subject.

2. The method of claim 1, wherein the CYT score is calculated according to the formula:
$$CYT = a - b ( cg 07786657 ) + c ( cg 12446199 ) - d ( cg 00027570 ) ,$$
wherein a is 0 to 10,
wherein b is 2 to 3,
wherein c is 0.5 to 1.5,
wherein d is 1 to 2.

3. The method of claim 2, wherein the CYT score is calculated according to the formula:
$$CYT = 6.31 - 2.11 \times ( cg 07786657 ) + 0.96 \times ( cg 12446199 ) - 1.28 \times ( cg 00027570 ) .$$

4. The method of claim 1, wherein the sample is a tumor tissue sample.

5. The method of claim 1, wherein the sample is a plasma sample.

6. The method of claim 1, wherein the immunotherapy agent is an immune checkpoint inhibitor.

7. The method of claim 6, wherein the checkpoint inhibitor comprises an anti-PD-1 antibody, anti-PD-L1 antibody, anti-CTLA-4 antibody, or a combination thereof.

8. A personalized method for treating a tumor a subject comprising:
(a) detecting reduced TCF7 levels (TCF7^{low}), LAG3 levels (LAG310w), HAVCR2 levels (HAVCR2^{low}), TIGIT levels (TIGIT^{low}), or a combination thereof, in a sample from the subject compared to a control, or detecting elevated methylation levels of cg25947408 in the sample from the subject indicative of lower TCF7 levels (TCF7^{low}), LAG3 levels (LAG3^{low}), HAVCR2 levels (HAVCR2^{low}), and/or TIGIT levels (TIGIT^{low}) in the tumor; and
(b) detecting in the sample methylation levels of cg07786657, cg12446199, and cg00027570, and calculating a high cytolytic activity score (CYT^{high}) score from the methylation levels; and
(c) administering an immunotherapy agent to the subject.

9. The method of claim 8, further comprising:
(b) detecting in the sample methylation levels of cg07786657, cg12446199, and cg00027570; and calculating a high cytolytic activity score (CYT^{high}) score from the methylation levels,
wherein a high CYT score (CYT^{high}) and low TCF7 levels (TCF7^{low}) is an indication that the tumor will respond to the immunotherapy agent.

10. The method of claim 8, wherein the CYT score is calculated according to the formula:
$$CYT = a - b ( cg 07786657 ) + c ( cg 12446199 ) - d ( cg 00027570 ) ,$$
wherein a is 0 to 10,
wherein b is 2 to 3,
wherein c is 0.5 to 1.5,
wherein dis 1 to 2.

11. The method of claim 9, wherein the CYT score is calculated according to the formula:
$$CYT = 6.31 - 2.11 \times ( cg 07786657 ) + 0.96 \times ( cg 12446199 ) - 1.28 \times ( cg 00027570 ) .$$

12. The method of claim 8, wherein the sample is a tumor tissue sample.

13. The method of claim 8, wherein the sample is a plasma sample.

14. The method of claim 8, wherein the immunotherapy agent is an immune checkpoint inhibitor.

15. The method of claim 14, wherein the checkpoint inhibitor comprises an anti-PD-1 antibody, anti-PD-L1 antibody, anti-CTLA-4 antibody, or a combination thereof.

16. The method of claim 1, wherein the tumor is melanoma, head and neck cancer, colon cancer, stomach cancer, lung adenocarcinoma, lung squamous cell carcinoma, uterine cancer, glioma, cervical cancer, breast cancer, bladder cancer or colorectal cancer.

17. A kit comprising detection probes or primers configured to detect methylation of cg07786657, cg12446199, cg00027570, and cg25947408.
